(19) United States
(12) Patent Application Publication
(10) Pub. No.: US 2010/0241164 A1
(43) Pub. Date: Sep. 23, 2010

(54) Facet joint implant

(75) Inventors: Kay Fischer, Tuttlingen (DE); Joerg Schumacher, Teltow (DE); Jens Beger, Tuttlingen (DE)

Correspondence Address: Lipsitz & McAllister, LLC, 755 MAIN STREET, MONROE, CT, 06468, US

(73) Assignee: Aesculap AG, Tuttlingen (DE)

(21) Appl. No.: 12/661,629

(22) Filed: Mar. 18, 2010

Related U.S. Application Data
(63) Continuation of application No. PCT/EP2008/008060, filed on Sep. 24, 2008.

(30) Foreign Application Priority Data
Oct. 30, 2007 (DE) 10 2007 051 783.3

(51) Int. Cl. A61B 17/70

(52) U.S. Cl. 606/247

(57) ABSTRACT

In a facet joint implant comprising two components adapted in the area of a facet-contacting surface for placement on the outer surface of a facet, and a fastener for joining the two components to each other through a channel in a facet or in two facets lying against each other, in order to facilitate insertion of the implant, it proposed that the fastener be configured as a flexible thread which tensions the two components towards each other such that the components are pressed against the outer surface of the facet against which they lie.

This application is a continuation of international application number PCT/EP2008/008060 filed on Sep. 24, 2008, and claims the benefit of German Patent Application No. 10 2007 051 783.3 filed on Oct. 30, 2007.

The present disclosure relates to the subject matter disclosed in international application number PCT/EP-2008/008060 of Sep. 24, 2008 and German application number 10 2007 051 783.3 of Oct. 30, 2007, which are incorporated herein by reference in their entirety and for all purposes.

### BACKGROUND OF THE INVENTION

The invention relates to a facet joint implant comprising two components adapted for placement in the area of a facet-contacting surface on the outer surface of a facet, and a fastener for joining the two components to each other through a channel in a facet or in two facets lying against each other.

The vertebral joints or facet joints often contribute to a large extent to back pain. Degenerative changes and wear of the joint surfaces may result in an increase in pressure on the nerve endings and therefore cause pain. The increasing instability of the facet joint may on account of use of compensation mechanisms bring about hypertrophy of the facet joint and result in spinal stenosis or foraminal stenosis. In this case, part of the lamina and the facet joint is often removed, and it is also standard practice to fuse the segment in order to prevent instability.

Damage to the facet joint often also prevents insertion of intervertebral disc prostheses as insertion of these can only be effective if the facet joints are intact.

It is known to replace facet joints by implants, and the implants are often held on the facets by complicated mechanical constructions. It is, for example, known to implant in the body facet joint implants consisting of two components which accommodate between them a facet or two facets lying against each other and which are connected to each other by rods or screws to which the two components are attached passing through a channel in the facet or the facets lying against each other (US 2006/0036323 A1; US 2005/0049705 A1; US 2005/0085912 A1; US 2006/0085075 A1). The connection by means of rods or screws makes insertion of the implant into the body difficult and, in addition, these connecting devices in the form of screws or rods are difficult to connect to the components. Tensioning of the components towards one another requires considerable space at the operating site and therefore leads to large access openings.

The object of the invention is to so construct a generic facet joint implant that its implantation and placement on the facet or facets are facilitated.

### SUMMARY OF THE INVENTION

This object is accomplished, in accordance with the invention, in a facet joint implant of the kind described at the outset in that the fastener is configured as a flexible thread which tensions the two components towards each other such that the components are pressed against the outer surface of the facet against which they lie.

Use of a flexible thread as fastener enables introduction of the implant components and the fastener through a very small access, and the tensioning of the two components towards each other can also be effected in a simple way by the thread joining the two components being drawn through a channel in the facet or in the two facets and tensioned. This requires only a small space in the area of the implant components as the thread can be deflected and the pulling forces then applied in any direction.

The thread may also be in the form of a wire or a cord or the like, it merely being essential that it be a flexible, thread-shaped pulling device. For example, a surgical suture thread could be used as thread.

In accordance with a preferred embodiment, provision is made for at least one of the components to be of such small dimensions that it is insertable through the channel in the facet or facets. In this way, implantation of the implant is possible from one side of a facet. Proceeding from this one side, the insertable component is first inserted through the channel in the facet as far as the opposite side of the facet where it is then arranged transversely to the channel so that this component acts as retaining element lying against the outer side of the facet. Between the facets, the thread then runs through the channel and can be tensioned, so that the two components on opposite sides of the channel are pulled against the outer surfaces of the facet.

Here it is advantageous for the insertable component to be a narrow, elongate component whose length is greater than the diameter of the channel. For example, the insertable component may have a substantially rectangular shape.

It is expedient for the insertable component to be bent so as to have a concave facet-contacting surface and a convex outer surface. As a result, this component positions itself with the facet-contacting surface with surface-to-surface contact on the outer side of the facet and also outwardly enlarges the facet only slightly owing to the convex outer surface.

In a particularly preferred embodiment, provision is made for a pull member to engage an end face of the insertable component. This may be, for example, one or more threads or also a needle which is joined to the insertable component, if necessary, also with a thread placed between these. This pull member can first be inserted through the channel in the facet, for example, with the aid of a needle holder, and the insertable component can then be made to follow the pull member through the channel in the facet. This makes it easier for the insertable component to be introduced and pushed through.

It is particularly advantageous for the thread joining the two components to run back and forth several times between the two components and to be displaceably deflected at least on one component so as to produce a pulley assembly. With relatively low pulling forces on the thread, high tension forces can thereby be generated, with which the two components of the facet joint implant are pressed against the outer surfaces of the respective facet.

Accordingly, it may be provided that at least one of the components has at least two adjacent openings as deflection for the thread.

In a preferred embodiment, at least one of the components has a central opening for the thread or threads.

It is expedient for at least one component to carry a centering projection which enters the channel when the component lies against the facet. The component is thereby secured against lateral displacement and guided into a precisely defined position in which it lies against the facet when it is tensioned against the facet.

In a preferred embodiment, the centering projection may be constructed as a sleeve which surrounds an opening in the component, through which the thread or threads are led.

It is expedient for the interior of the sleeve to be in communication with at least one thread channel radiating radially from the sleeve. In this way, the thread can run in the plane of the facet joint surfaces and thereby be led to the outside.

In a first preferred embodiment, provision is made for the component adapted for placement on the inner side of a facet to be constructed as facet joint surface on its side opposed to the facet-contacting surface. This component can thus replace a joint surface of the facet joint.

Such an implant can only be arranged on one facet, so that only one joint surface of the facet is replaced. It is, however, also possible to arrange such implants on both facets belonging to a facet joint, so that the inwardly lying components then replace the two joint surfaces of the facet joint and lie against each other. These components then preferably lie in an articulated manner against each other, in particular, it may be provided that the facet joint surface of the inwardly lying component has a spherical depression or a spherical elevation for placement on a spherical elevation or a spherical depression, respectively, of a facet joint surface of a second facet joint implant of the same facet joint.

The components of the implant may also lie against the outer sides of two facets which lie against each other on their inner side via a natural facet joint. In this case, the two facets are permanently tensioned towards each other by such an implant in such a way that fusion of the facets in the joint area is made possible.

In a preferred embodiment, provision is made for the component adapted for placement on the outer side of a facet to be surrounded by a frame on which the component is supported and by which the contacting surface of the component on the outer side of the facet is enlarged. This frame may have very different shapes, it merely being essential that the component is supported on it and that the contacting surface on the facet is enlarged by the frame.

For example, the frame may surround the component completely.

It is also possible for the frame to have a through hole at the side, through which the thread or threads joining the two components to each other can pass. In this way it is possible to fit in the frame after insertion of the implant.

It may also be provided that the frame has a depression accommodating the component, so that the frame and the component inserted in the depression form a component for supporting the implant on the outer side of the facet, whose outer contour forms a unit. Injury to the surrounding tissue is thereby avoided to a large extent.

The component adapted for placement on the inner side of a facet may carry fixation projections, for example, in the form of tips, spikes or ribs, on its facet-contacting surface. Preferably, the ribs on a disc-shaped component can extend radially outwardly. These projections secure the component against displacement or turning relative to the facet as they penetrate the bone surface slightly.

In a preferred embodiment, provision is made for the thread to form a loop in the area between the two components and for the ends of the thread to be knotted by a tension knot displaceable on one end of the thread. As a result, the thread can be tensioned in a manner known per se by this knot being displaced on the end of the thread, for example, by means of a knot pusher as used when suturing a wound using a surgical suture thread.

The following description of preferred embodiments serves for a more detailed explanation in conjunction with the drawings.

### BRIEF DESCRIPTION OF THE DRAWINGS

FIG. 1 shows a perspective representation of two vertebrae with a facet joint implant for replacement of a facet joint surface;

FIG. 2 shows an enlarged detailed view of area A in FIG. 1;

FIG. 3 shows a perspective view of the facet joint implant of FIG. 2 with a joint surface component, a counter holder component, and a thread led in the manner of a pulley for joining the two components;

FIG. 4 shows a perspective view of the implant of FIG. 3 seen from the joint surface side;

FIG. 5 shows a perspective view of the facet-contacting surface of a joint surface component with projections on the facet-contacting surface;

FIG. 6 shows a view similar to FIG. 2 of implants for replacement of both joint surfaces of a facet joint;

FIG. 7 shows a perspective view of the two implants of FIG. 6 prior to their mutual engagement;

FIG. 8 shows a view similar to FIG. 3 of a modified embodiment with a needle-shaped pull member on the counter holder component;

FIG. 9 shows a view of a facet joint implant according to FIG. 3 with a frame surrounding the counter holder component;

FIG. 10 shows a view similar to FIG. 9 with a frame displaced in relation to the counter holder component; and

FIG. 11 shows a view similar to FIG. 2 with a facet joint implant tensioning two facets towards each other.

### DETAILED DESCRIPTION OF THE INVENTION

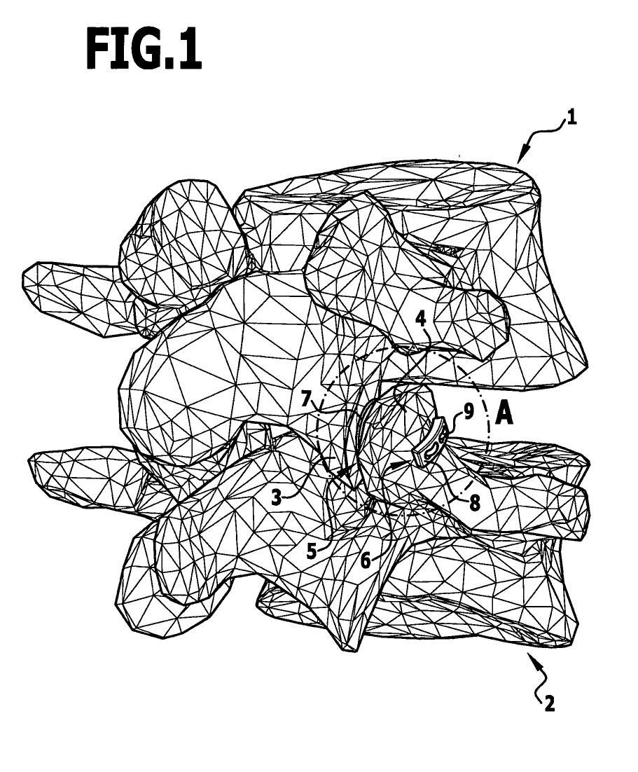

Two adjacent vertebrae 1, 2 are shown in FIG. 1. The vertebrae each carry two superior and two inferior bony projections which are referred to as facets 3, 4. An inferior facet 3 of an upper vertebra 1 and a superior facet 4 of a lower vertebra 2 lie against each other, in each case, and form a facet joint 5 in the area of contact. In the area of the facet joint, the two facets 3, 4 carry joint surfaces, which are almost plane and only have a slight curvature. They are each normally covered with a layer of cartilage, so that the layers of cartilage of the joint surfaces lie with surface-to-surface contact against one another in the area of the facet joint 5.

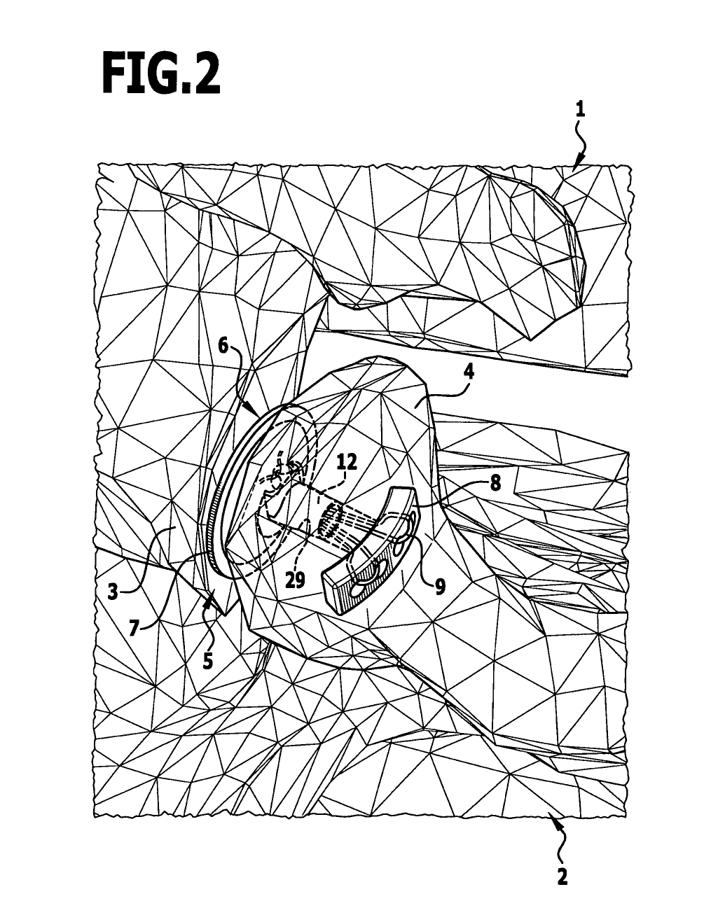

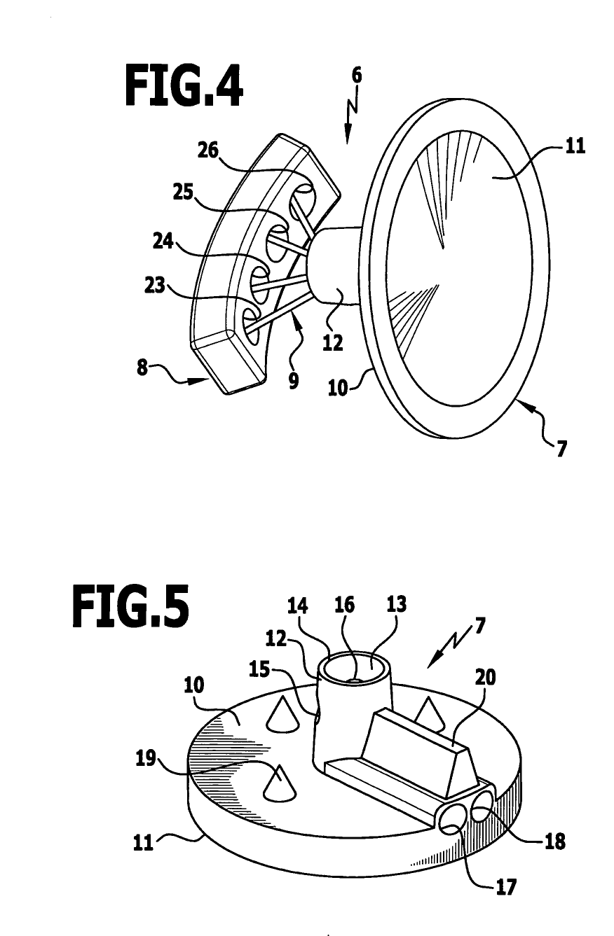

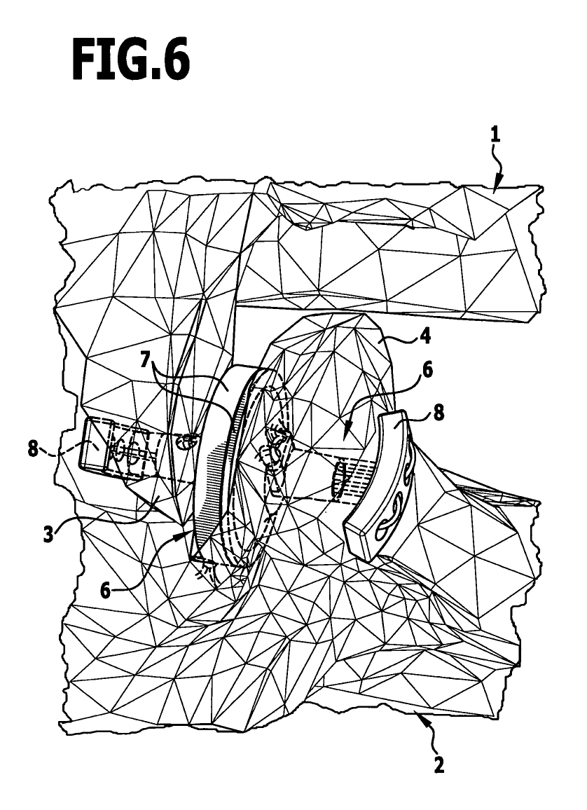

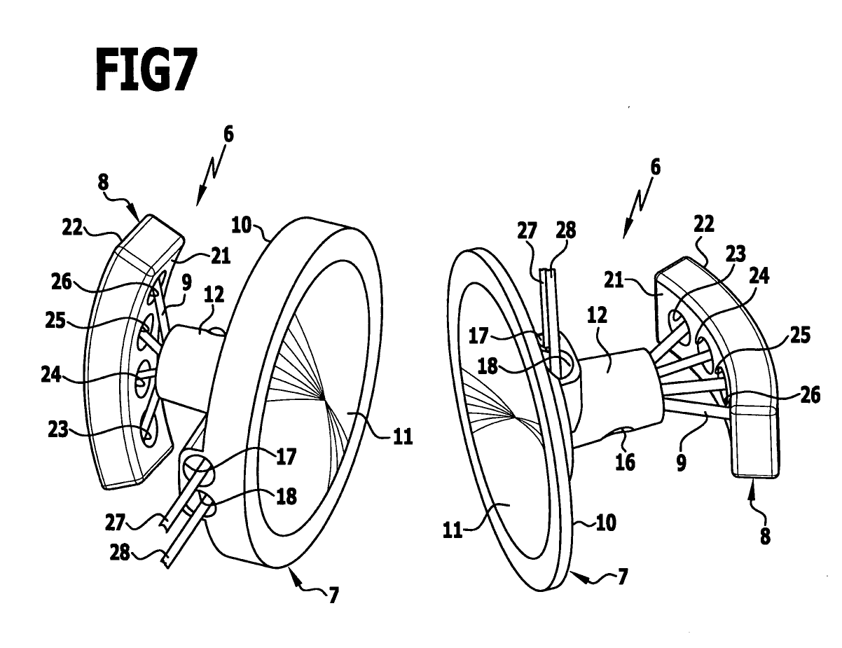

In the embodiment shown in FIGS. 1 and 2, the joint surface of a superior facet 4 of a lower vertebra 2 is replaced by a facet joint implant 6 comprising two components 7, 8, which are joined to each other by a thread 9. The first component 7 is arranged on the inner side of the facet 4 and replaces the natural facet joint surface on this inner side of the facet 4. To this end, the component 7 has the shape of a disc of circular cross section, which with a facet-contacting surface facing the facet 4 lies in the area of the natural facet joint surface against the facet 4, thereby covering the natural facet joint surface. The outer surface of the disc opposed to the facet-contacting surface 10 is constructed as facet joint surface 11. This facet joint surface 11 may, for example, have a slightly spherical depression, so that the natural joint surface of the opposite facet 3 or a similar facet joint surface 11 of an identically constructed facet joint implant on the other facet 3, in which the joint surface is complementarily of spherically elevated construction, lies with surface-to-surface contact against the joint surface and thereby forms a partially or completely replaced facet joint. In the embodiment of FIGS. 1 to 4, only one facet joint surface is replaced in each case by such a facet joint implant. In contrast, in the embodiment of FIGS. 6 and 7 each of the two facets lying against each other at a facet joint carries such a facet joint implant. Except for the curvature of the facet joint surfaces 11 these are of identical construction. The two joint surfaces are of complementary arched construction, so that they fit into each other with surface-to-surface contact, as will be clearly apparent from the representation of FIG. 7.

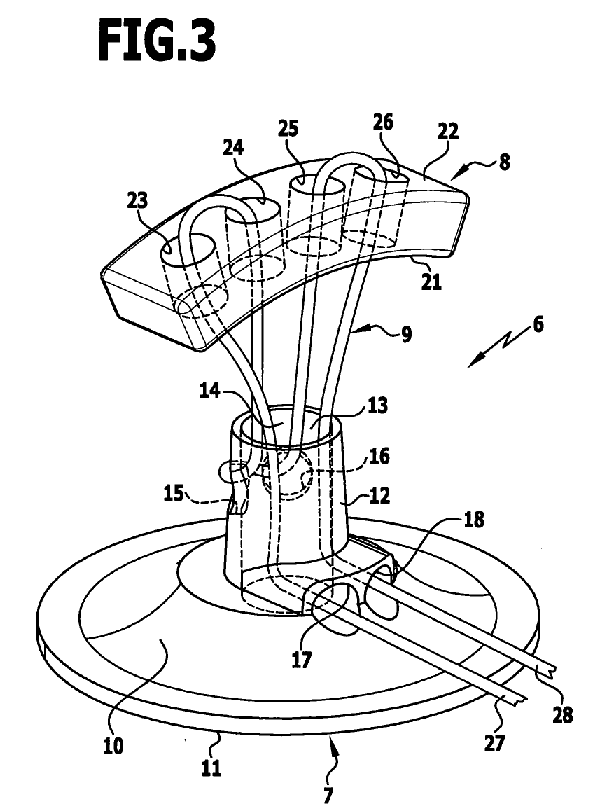

On the facet-contacting surface 10, the component 7 carries a central sleeve 12 extending with its longitudinal axis perpendicularly to the disc-shaped component 7. Its interior 13 is closed at one side by the disc-shaped component 7, whereas the opposite side is open. Two adjacent through holes 15 and 16 are arranged in the side wall 14 of the sleeve 12. Also, two adjacent, radially extending thread channels 17, 18 enter the sleeve 12 at its closed end. In the embodiment of FIG. 3, these are of relatively short construction, so that they terminate at a spacing within the rim of the disc-shaped component 7. In the embodiment of FIG. 5, in contrast, these thread channels 17, 18 terminate at the outer edge of the disc-shaped component 7.

Also, fixation projections can be provided on the facet-contacting surface 10, for example, in the form of tips or spikes 19 or in the form of a rib 20, which, in the embodiment of FIG. 5, extends above the two thread channels 17, 18 parallel thereto between the sleeve 12 and the outer rim of the disc-shaped component 7.

Such fixation projections may also be provided on the other components of the facet joint and on the other embodiments of these components.

The second component 8 of the facet joint implant 6 has the shape of an elongate rectangle or cuboid which is bent in the longitudinal direction, thereby producing a concave, inwardly lying facet-contacting surface 21 and a convex outer surface 22. The curvature of the two components 8 is so selected that when the facet-contacting surface 21 lies against the outer side of a facet it lies with surface-to-surface contact thereon, i.e., it corresponds to the curvature of a facet in the area of contact.

In the embodiments shown in the drawings, the second cuboidal component 8 carries four adjacent through openings 23, 24, 25, 26, through which the thread 9 is led. In the embodiment shown, this thread 9 passes, in order to join the two components 7, 8, through a thread channel 17 into the interior 13 of the sleeve 12 and is then led in the opposite direction through two adjacent openings 23, 24 of the second component 8. The thread 9 is deflected at this second component 8 and lies between the two openings 23, 24 against the outer surface 22 of the second component 8.

From the second opening 24 the thread 9 passes through the interior 13 of the sleeve 12 from the inside to the outside through the through hole 15 and then through the adjacent through hole 16 into the interior 13 of the sleeve 12 again. As it continues its course, the thread runs in the opposite direction again through the two openings 25 and 26 of the second component 8 and from the opening 26 finally through the interior 13 of the sleeve 12 and the second thread channel 18 reaches the outside again. The thread 9 thus forms two adjacent loops. When the ends 27, 28 of the thread 9 are pulled, this causes the thread 9 to tighten and pull the two components 7, 8 towards each other in the manner of a pulley.

To insert the described facet joint implant 6, a channel 29 extending substantially perpendicularly to the natural facet joint surface is first drilled through the corresponding facet 4. The facet joint capsule is opened, and the facet joint surface is prepared, for example, with a rasp. For accelerated fusion, it is also possible to completely remove the cartilage layer of the facet joints. Furthermore, the drilled channel can be filled with growth factors, for example, BMPs (bone morphogenetic proteins=proteins for the formation of bone). Through the channel 29 from the inner side of the facet, i.e., from the facet joint side, the second component 8 of a facet joint implant 6 is inserted through the channel 29 until it emerges from this channel 29 on the outer side of the facet. During this, the thread 9 is loose so that the second component 8 can be inserted through the channel 29 while the first component 7 still remains outside the facet joint space.

Then, after spreading out the facet joint space the first component 7 is introduced into the space between the two facets 3, 4, so that the sleeve 12 enters the channel 29. The sleeve 12 therefore acts as centering projection which by penetrating the channel 29 defines and fixes the position of the first component 7. After insertion through the channel 29, the second component 8 is tilted and arranged such that it covers the exit of the channel 29 and such that it places itself with its facet-contacting surface 21 against the outer side of the facet 4.

In order to secure the two components 7, 8 to the facet, the thread 9 is now tensioned. This can be done either by the thread being pulled out at both ends 27, 28 relative to the second component 8 or by one end 27 being knotted to the other end 28 in such a way that the resulting knot is freely displaceable at one end. By displacing the knot in the direction towards the second component 8 and by simultaneously pulling the other end, the thread 9 can be tensioned in the area between the two components 7, 8, and this tensioning is then maintained by the knot. When tensioning the thread, the two components 7, 8 are pressed in each case against the facet 4, and the tips 19 and the rib 20 thereby press into the facet 4 and secure the disc-shaped component 7 against any displacement relative to the facet 4. Solely by tensioning the thread, the two components 7, 8 are thus firmly and permanently secured to the facet 4.

If the facet joint 5 is only replaced at one joint surface, only one such implant is secured to a facet. If both joint surfaces are to be replaced, a corresponding implant is secured in each case to each facet. The facet joint surfaces of the two implants then position themselves on each other in an articulated manner and with surface-to-surface contact, as will be explained with reference to FIG. 7.

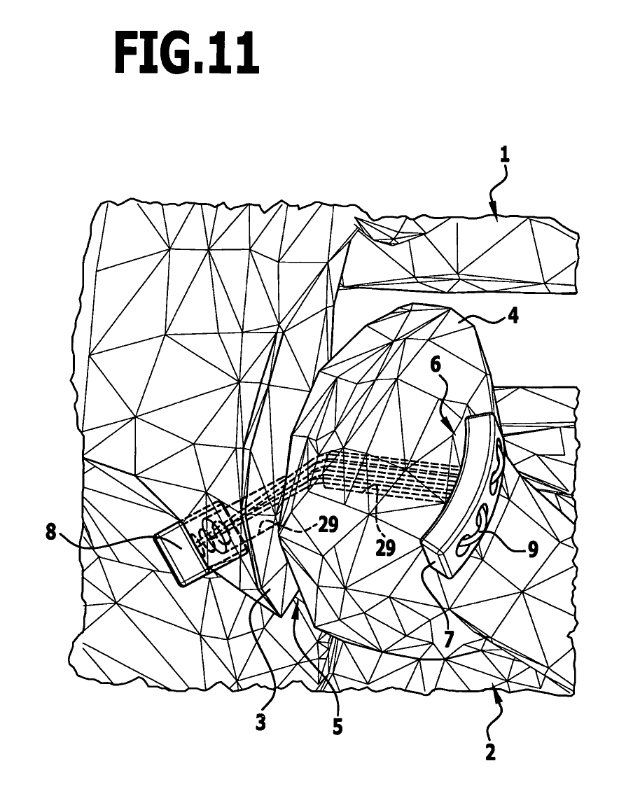

If the two facets 3, 4 are to be permanently fixedly joined to each other, i.e., if a fusion of the two facets is desired, then a corresponding channel 29 is drilled in both facets, with the channels in alignment with each other, and a similar facet joint implant 6 is then inserted, which, however, differently from the facet joint implant described hereinabove, has two components which are both identical in design to the second component 8 of the facet joint implant of FIGS. 1 to 4. Accordingly, a component which is of disc-shaped construction and replaces a joint surface is then missing in this implant. Both components are constructed as pure holding elements like the outwardly lying component 8 of the embodiment of FIGS. 1 to 4. These engage the outer side of the facets and when the thread is tensioned then press the two facets permanently against each other, as shown in FIG. 11.

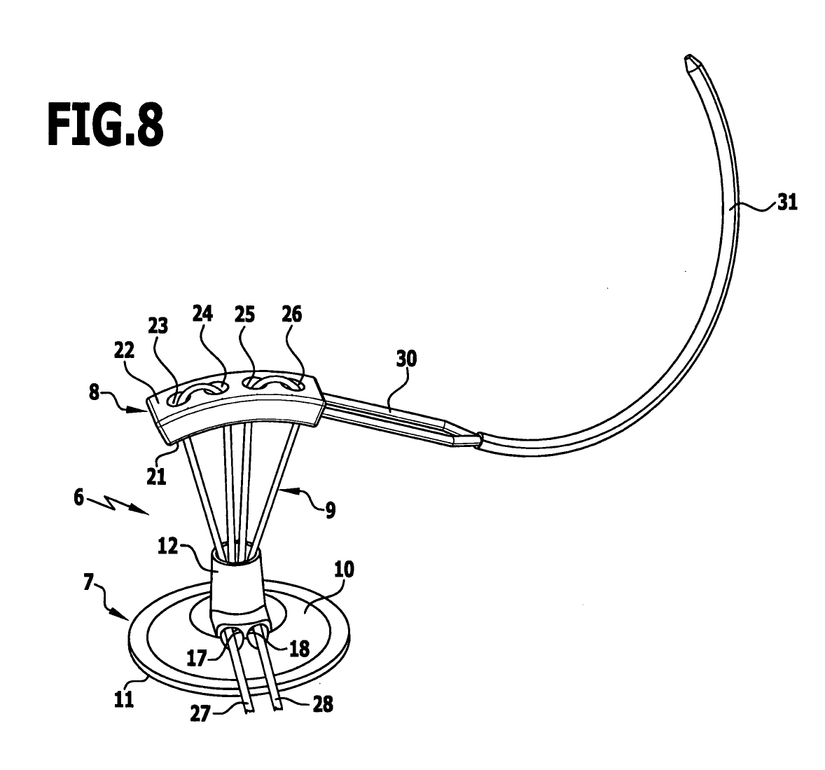

The insertion of the elongate, rectangular, outwardly lying component 8 is then facilitated by arrangement, on an end face thereof, of a thread 30 and a preferably bent needle 31, as shown in FIG. 8. Upon insertion, for example; with the aid of a needle holder, the needle 31 is first pulled from the facet joint side through the channel 29, and the thread 30 joined to the needle 31 then pulls the elongate component 8 behind it through the channel 29, so that the surgeon has no difficulty in introducing the component 8 on the joint side into the channel 29.

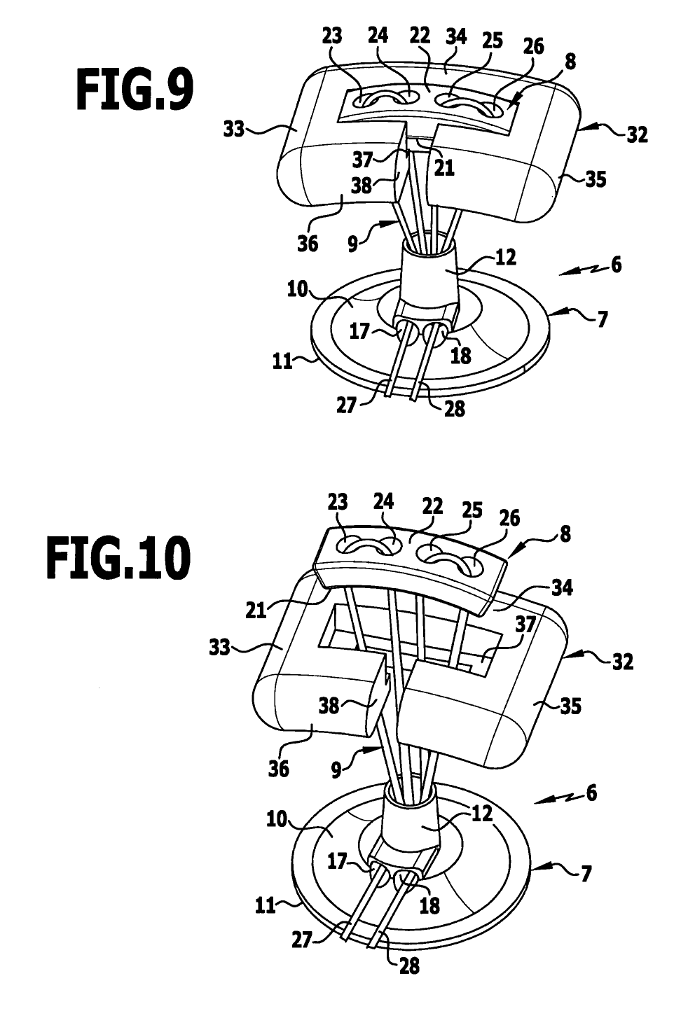

The cross-sectional area of the outwardly lying second component 8 is normally chosen so as to be relatively small in order that this outwardly lying component 8 will be able to be inserted through the channel 29 of the facet. This may result in relatively small pressure surfaces on the outer side of the facet. In order to enlarge these contacting surfaces, a frame 32 may be provided, which surrounds the component 8 and thereby increases its contacting surface on the facet. This frame 32 may, for example, as shown in FIG. 9, comprise outwardly lying legs 33, 34, 35, 36, which surround a depression 37 which itself forms an accommodation space for the outwardly lying second component 8. At the side, this depression 37 can form in the area of a leg 36 a gap 38 through which the thread 9 can be led when the component 8 is inserted from above into the depression 37. In the case of such a frame, after insertion of the facet joint implant in the above-described manner and before tensioning the thread, this frame on the outer side of the facet is pushed at the sides up to the thread 9 and the outwardly lying component 8 so that insertion of the component 8 into the depression 37 is made possible and this component 8 fills out this depression fully after insertion, as shown in FIG. 9. Upon tensioning the thread, the frame 32 with the outwardly lying component 8 accommodated therein is then pulled against the outer side of the facet 4, and the contacting surface on the outer side of the facet is thereby enlarged in relation to the relatively small contacting surface of the outwardly lying component 8 alone.

The frame 32 may, of course, also have different shapes, for example, the shape of a somewhat larger plate.

There are cases where parts of the facet have to be resected, and the frame 32 can then serve to fill out a defect. In this case, the frame is preferably constructed in accordance with the shape of the defect and inserted into it, so that the missing bone material of the facet is replaced by the frame.

In this case, the frame can, for example, stabilize the facet joint to withstand bending loads.

Here it is advantageous for the frame to consist of a bone-compatible material, for example, a metal, a coated metal or a polymer, or to also have a porous metal structure into which the bone can grow. In this way, the frame assumes a double function, firstly as defect filling and secondly as counter bearing for the thread 9 for tensioning the two components 7, 8 of the facet joint 6 towards each other.

The components may be made from metal, ceramic or polymers, for example, from polyetheretherketone (PEEK), from polyethylene (PE) or from carbon fiber-reinforced polyetheretherketone (CFR PEEK). The polymers may have an osteoconductive coating of metal or ceramic so as to promote the connection to the adjacent bone material.

Conceivable combinations of materials for the sliding surfaces of the facets are:
metal/polymer: e.g. CoCroMo/ultrahigh molecular weight polyethylene (UHMWPE)
   CoCroMo/carbon fiber-reinforced polyethylene (CFR PEEK)
polymer/polymer: e.g. CFR PEEK/CFR PEEK
ceramic/ceramic: e.g. titanium ceramic-coated/titanium ceramic-coated
   hydrogel coatings on metal or polymer.

1. Facet joint implant comprising two components adapted in the area of a facet-contacting surface for placement on the outer surface of a facet, and a fastener for joining the two components to each other through a channel in a facet or in two facets lying against each other, wherein the fastener is configured as a flexible thread which tensions the two components towards each other such that the components are pressed against the outer surface of the facet against which they lie.

2. Facet joint implant in accordance with claim 1, wherein at least one of the components is of such small dimensions that it is insertable through the channel in the facet or facets.

3. Facet joint implant in accordance with claim 2, wherein the insertable component is a narrow, elongate component whose length is greater than the diameter of the channel.

4. Facet joint implant in accordance with claim 3, wherein the insertable component has a substantially rectangular shape.

5. Facet joint implant in accordance with claim 3, wherein the insertable component is bent so as to have a concave contacting surface for placement on the facet and a convex outer surface.

6. Facet joint implant in accordance with claim 2, wherein a pull member engages an end face of the insertable component.

7. Facet joint implant in accordance with claim 6, wherein the pull member comprises one or more threads.

8. Facet joint implant in accordance with claim 7, wherein the pull member comprises a needle.

9. Facet joint implant in accordance with claim 1, wherein the thread joining the two components runs back and forth several times between the two components and is displaceably deflected at least on one component so as to produce a pulley assembly.

10. Facet joint implant in accordance with claim 9, wherein at least one of the components has at least two adjacent openings as deflection for the thread.

11. Facet joint implant in accordance with claim 1, wherein at least one of the components has a central opening for the thread or threads.

12. Facet joint implant in accordance with claim 1, wherein at least one component carries a centering projection which enters the channel when the component lies against the facet.

13. Facet joint implant in accordance with claim 12, wherein the centering projection is constructed as a sleeve surrounding an opening in the component, through which the thread or threads are led.

14. Facet joint implant in accordance with claim 13, wherein the interior of the sleeve is in communication with at least one thread channel radiating radially from the sleeve.

15. Facet joint implant in accordance with claim 1, wherein the component adapted for placement on the inner side of the facet is constructed as facet joint surface on its side opposed to the facet-contacting surface.

16. Facet joint implant in accordance with claim 15, wherein the facet joint surface has a spherical depression or a spherical elevation for placement on a spherical elevation or a spherical depression, respectively, of a facet joint surface of a second facet joint implant.

17. Facet joint implant in accordance with claim 1, wherein the component adapted for placement on the outer side of a facet is surrounded by a frame on which the component is supported and by which the contacting surface of the component on the outer side of the facet is enlarged.

18. Facet joint implant in accordance with claim 17, wherein the frame has a through hole at the side.

19. Facet joint implant in accordance with claim 17, wherein the frame has a depression accommodating the component.

20. Facet joint implant in accordance with claim 1, wherein the component adapted for placement on the inner side of a facet carries fixation projections on its facet-contacting surface.

21. Facet joint implant in accordance with claim 20, wherein the fixation projections are in the form of tips.

22. Facet joint implant in accordance with claim 20, wherein the fixation projections are in the form of ribs.

23. Facet joint implant in accordance with claim 1, wherein the ends of the thread are knotted by a tension knot displaceable on one end of the thread.
